(19) United States
(12) Patent Application Publication
(10) Pub. No.: US 2018/0214128 A1
(43) Pub. Date: Aug. 2, 2018

(54) METHOD AND ULTRASOUND IMAGING SYSTEM FOR REPRESENTING ULTRASOUND DATA ACQUIRED WITH DIFFERENT IMAGING MODES

(71) Applicant: General Electric Company, Schenectady, NY (US)

(72) Inventor: Branislav Holländer, Vöcklabruck (AT)

(21) Appl. No.: 15/440,215

(22) Filed: Feb. 23, 2017

Related U.S. Application Data
(63) Continuation-in-part of application No. 15/420,192, filed on Jan. 31, 2017, now abandoned.

(51) Int. Cl. A61B 8/00; A61B 8/06; A61B 8/14; A61B 8/08

(52) U.S. Cl. CPC A61B 8/463; A61B 8/06; A61B 8/467; A61B 8/5207; A61B 8/14

(57) ABSTRACT

A method and ultrasound imaging system for displaying ultrasound includes acquiring first ultrasound data for a plane while in a first ultrasound imaging mode and acquiring second ultrasound imaging data for the plane while in a second ultrasound imaging mode. The first ultrasound data comprises a first plurality of values and the second ultrasound data comprises a second plurality of values. The method and system includes generating a surface-rendering based on both the first and second ultrasound data, where the surface-rendering comprises a non-planar surface. The first ultrasound data is represented by one of a plurality of colors and a plurality of grey-scale values in the surface-rendering, while the second ultrasound data is represented by a plurality of heights of the non-planar surface in a Z-direction. The method and system includes displaying the surface-rendering on a display device.

### CROSS REFERENCE TO RELATED APPLICATIONS

This application is a Continuation-In-Part of U.S. patent application Ser. No. 15/420,192, entitled “METHOD AND ULTRASOUND IMAGING SYSTEM FOR REPRESENTING ULTRASOUND DATA ACQUIRED WITH DIFFERENT IMAGING MODES”, filed Jan. 31, 2017, which is herein incorporated by reference.

### FIELD OF THE INVENTION

This disclosure relates generally to an ultrasound imaging system and method for generating and displaying a surface-rendering to represent two different modes of ultrasound data at the same time.

### BACKGROUND OF THE INVENTION

It is known to use ultrasound imaging systems to acquire ultrasound imaging data while in different ultrasound imaging modes. Various ultrasound imaging modes may be used to acquire ultrasound data for different parameters, which may be used to provide different types of information to a clinician. Examples of different ultrasound imaging modes that are commonly used include B-mode, strain, strain rate, and color Doppler. It is challenging to display ultrasound data from more than one ultrasound imaging mode. Conventional techniques for color Doppler imaging replace B-mode pixel values with colors to show the direction and velocity of flow. However, the color values typically overwrite the B-mode values, which makes the overwritten B-mode data more difficult to interpret. Additionally, it can be challenging for the clinician to differentiate small differences in the flow data (or any other type of data) when using color, as it is difficult for many users to reliably detect small differences in the colors used to represent the values associated with the flow data, or any other type of data represented with the color.

For these and other reasons, an improved method and ultrasound imaging system for generating and displaying ultrasound imaging data acquired with two different ultrasound imaging modes is desired.

### BRIEF DESCRIPTION OF THE INVENTION

The above-mentioned shortcomings, disadvantages and problems are addressed herein which will be understood by reading and understanding the following specification.

In an embodiment, a method of displaying data acquired from multiple ultrasound imaging modes includes acquiring first ultrasound data for a plurality of location within a lane while in a first ultrasound imaging mode, the first ultrasound data comprising a first plurality of values and acquiring second ultrasound data for the plurality of locations within the plane while in a second ultrasound imaging mode that is different than the first ultrasound imaging mode, the second ultrasound data comprising a second plurality of values. The method includes generating a surface-rendering based on both the first ultrasound data and the second ultrasound data. The surface-rendering comprising a non-planar surface and representing an X-direction, a Y-direction, and a Z-direction, where the first ultrasound data is represented by one of a plurality of colors and a plurality of grey-scale values in the surface-rendering, and where the second ultrasound data is represented by a plurality of heights of the non-planar surface in the Z-direction of the surface-rendering, and displaying the surface-rendering on a display device.

In an embodiment, an ultrasound imaging system includes a probe, a display device, and a processor in electronic communication with the probe and the display device. The processor is configured to control the probe to acquire first ultrasound imaging data for a plurality of locations within a plane while in a first ultrasound imaging mode, the first ultrasound data including a first plurality of values. The processor is configured to acquire second ultrasound imaging data for the plurality of locations within the plane while in a second ultrasound imaging mode that is different than the first ultrasound imaging mode, the second ultrasound data including a second plurality of values. The processor is configured to generate a surface-rendering to represent both the first ultrasound data and the second ultrasound data. The surface rendering including a non-planar surface and representing an X-direction, a Y-direction, and a Z-direction. Each of the plurality of locations is represented by a coordinate location in the surface-rendering in the X-direction and the Y-direction. The first ultrasound data is represented by one of a plurality of color values and a plurality of grey-scale values in the surface-rendering. The second ultrasound data is represented by a plurality of heights of the non-planar surface in the Z-direction of the surface-rendering. The processor is configured to display the surface-rendering on the display device.

### BRIEF DESCRIPTION OF THE DRAWINGS

FIG. 1 is a schematic diagram of an ultrasound imaging system in accordance with an embodiment;

FIG. 2 is a flow chart in accordance with an embodiment;

FIG. 3 is a schematic representation of a plane, a schematic representation of a perspective view of a plane, and a schematic representation of a technique for surface-rendering elements to represent values from multiple different imaging modes in accordance with an exemplary embodiment;

FIG. 4 is a schematic representation of a surface-rendering in accordance with an exemplary embodiment;

FIG. 5 is a schematic representation of a surface-rendering in accordance with an exemplary embodiment;

FIG. 6 is a schematic representation of a surface-rendering in accordance with an exemplary embodiment; and

FIG. 7 is a schematic representation of a surface-rendering in accordance with an exemplary embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

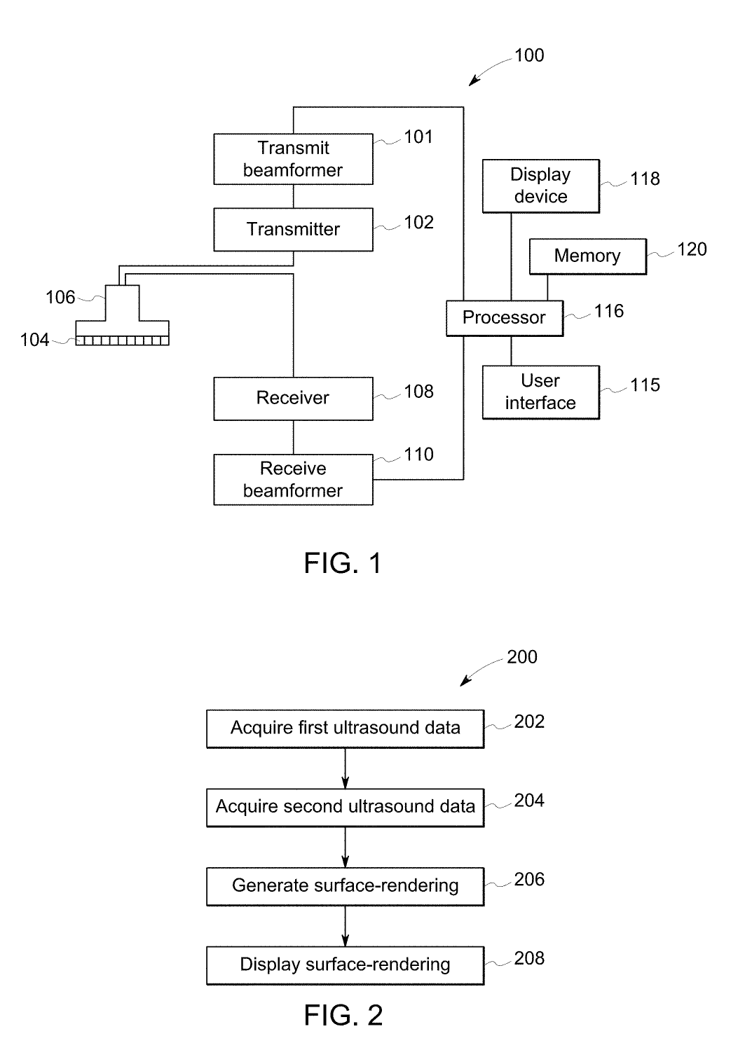

FIG. 1 is a schematic diagram of an ultrasound imaging system 100 in accordance with an embodiment. The ultrasound imaging system 100 includes a transmit beamformer 101 and a transmitter 102 that drive transducer elements 104 within a probe 106 to emit pulsed ultrasonic signals into a body (not shown). Still referring to FIG. 1, the pulsed ultrasonic signals are back-scattered from structures in the body, like blood cells or muscular tissue, to produce echoes that return to the transducer elements 104. The echoes are converted into electrical signals, or ultrasound data, by the transducer elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface may be used to control the input of patient data, or to select various ultrasound imaging modes, operations, and parameters, and the like. The user interface 115 may include a one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108 and the receive beamformer 110. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The beamformer 110 may be configured to perform conventional beamforming techniques as well as techniques such as retrospective transmit beamforming (RTB).

The processor 116 is in electronic communication with the probe 106. The processor 116 may control the probe 106 to acquire ultrasound data. The processor 116 controls which of the transducer elements 104 are active and the shape of a beam emitted from the probe 106. The processor 116 is also in electronic communication with a display device 118, and the processor 116 may process the ultrasound data into images for display on the display device 118. The images displayed on the display device 118 may comprise surface-renderings, for instance. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU) or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities. The data may be processed in real-time during a scanning session as the echo signals are received. For the purposes of this disclosure, the term “real-time” is defined to include a procedure that is performed without any intentional delay. Real-time frame or volume rates may vary based on the size of the region or volume from which data is acquired and the specific parameters used during the acquisition. The data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to display as an image. It should be appreciated that other embodiments may use a different arrangement of processors. For embodiments where the receive beamformer 110 is a software beamformer, the processing functions attributed to the processor 116 and the software beamformer hereinabove may be performed by a single processor such as the receive beamformer 110 or the processor 116. Or, the processing functions attributed to the processor 116 and the software beamformer may be allocated in a different manner between any number of separate processing components.

According to an embodiment, the ultrasound imaging system 100 may continuously acquire ultrasound data at a frame-rate of, for example, 10 Hz to 30 Hz. Images generated from the data may be refreshed at a similar frame-rate. Other embodiments may acquire and display data at different rates. For example, some embodiments may acquire ultrasound data at a frame rate of less than 10 Hz or greater than 30 Hz depending on the size of the volume and the intended application. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 to acquire ultrasound data in various ultrasound imaging modes (e.g., B-mode, Color Doppler, M-mode, Color M-mode, spectral Doppler, Elastography, TVI, strain, strain rate, and the like) to form 2D or 3D images or data. For example, one or more modules may generate B-mode, color Doppler, M-mode, color M-mode, spectral Doppler, Elastography, TVI, strain, strain rate and combinations thereof, and the like. Surface-renderings may be generated to display data according to surface-rendering techniques. Surface-rendering is a technique to represent surface data points in a manner to conveys the three-dimensionality of a surface defined by the relative three-dimensional positions of a plurality of surface data points. A surface-rendering may involve calculating shading, reflections and light scattering from a plurality of surface data points to accurately convey the contours and positions of the various surfaces formed by the surface data points. It is possible to visualize more information in a surface-rendering compared to a conventional two-dimensional image.

The image beams and/or frames are stored and timing information indicating a time at which the data was acquired in memory may be recorded. The modules may include, for example, a scan conversion module to perform scan conversion operations to convert the image frames from Polar coordinates to Cartesian coordinates. A video processor module may be provided that reads the image frames from a memory and displays the image frames in real time while a procedure is being carried out on a patient. A video processor module may store the image frames in an image memory, from which the images are read and displayed.

FIG. 2 is a flow chart of a method 200 in accordance with an exemplary embodiment. The individual blocks of the flow chart represent steps that may be performed in accordance with the method 200. Additional embodiments may perform the steps shown in a different sequence and/or additional embodiments may include additional steps not shown in FIG. 2. The technical effect of the method 200 is the display of a surface-rendering to display both the first ultrasound data and the second ultrasound data where the second ultrasound data was acquired with an ultrasound imaging mode that is different than the first ultrasound data.

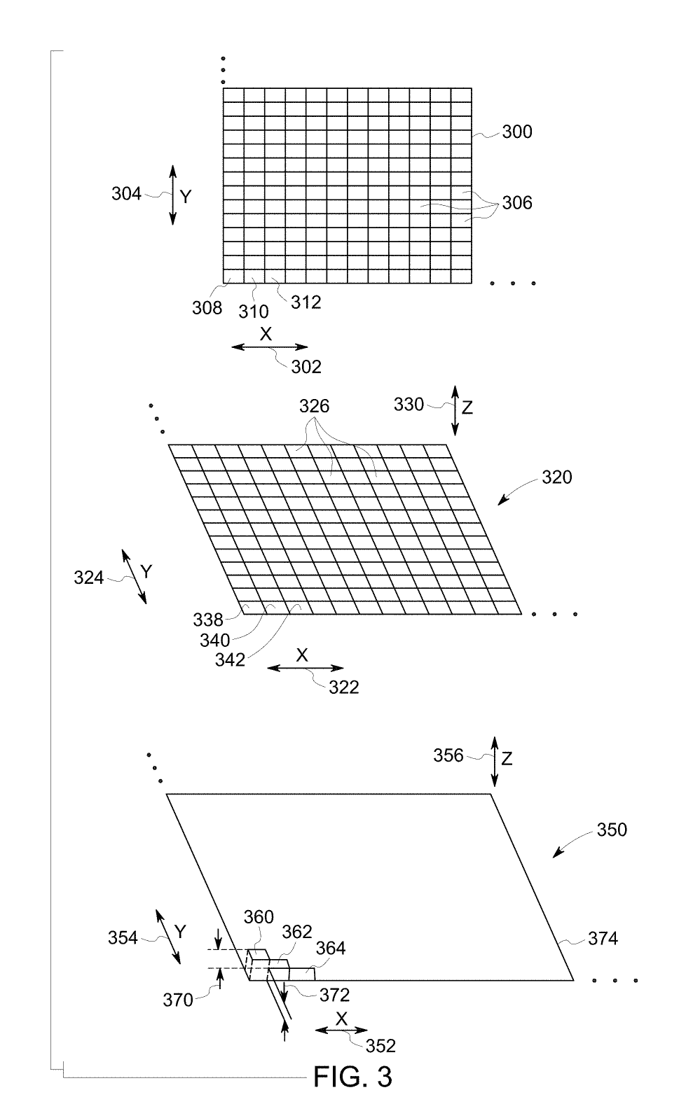

FIG. 3 is a schematic representation of a plane 300, a schematic representation of a perspective view of a plane 320, and a schematic representation of a technique for surface-rendering elements to represent values from multiple different imaging modes in accordance with an embodiment. The plane 300, has a X-direction 302 and a Y-direction 304. The plane 300 includes a plurality of locations 306. Each location 306 represents the location to which a value is assigned in the ultrasound data. The value assigned to each of the plurality of locations 306 may either be directly acquired for that location (i.e., the position of the location may correspond with a sample point on an ultrasound beam or line), or the value for one or more of the locations 306 may be interpolated based on the positions of sample points that were directly acquired. The process of assigning values to a plurality of locations within a plane is well-known within ultrasound and may, for instance, take place during scan conversion when converting ultrasound data from Polar coordinates to Cartesian coordinates for display on a display device such as the display device 118. Each of the plurality of locations 306 may be identified by a coordinate in the X-direction 302 and the Y-direction 304 of the plane 300. Collectively, the plurality of locations 306 define the plane 300 from which ultrasound data were acquired according to an embodiment.

The plane 300 includes the plurality of locations 306 for which ultrasound data are acquired. According to an embodiment, a value of a parameter may be acquired for each of the plurality of locations 306. The plane 300 includes a first location 308, a second location 310, and a third location 312. Only a subset of the total number of locations 306 in the plane 300 are schematically represented in FIG. 3. According to many embodiments, the plane 300 contains additional locations and extends further in both the X-direction 302 and the Y-direction 304.

FIG. 3 includes a schematic representation of a perspective view of a plane 320. The perspective view of the plane 320 is an example of a surface-rendering and may be generated by any known surface-rendering technique. The surface-rendering includes a representation a surface defined by a plurality of surface data points acquired from the plane 300. A location in three-dimensional space is acquired for each of the surface data points. A surface data points may be acquired from each of the plurality of locations 306 in the plane 300. The perspective view of the plane 320 includes an X-direction 322, a Y-direction 324, and a plurality of elements 326. Each of the elements 326 may be represented by one or more pixels according to various embodiments. The perspective view of the plane 320 includes a first element 338, a second element 340, and a third element 342. The elements in the perspective view of the plane 320 correspond to the locations 306 in the plane 300. Each of the locations in the plane 300 has a coordinate location in the X-direction 302 and the Y-direction 304. Each element in the perspective view of the plane (which is a surface-rendering) corresponds with one of the locations 306 in the plane. For example, the first location 308 corresponds with the first element 338; the second location 310 corresponds with the second element 340; and the third location 312 corresponds with the third element 322. In a similar manner, each element 326 in the perspective view of the plane 320 represents the data acquired from the location in the plane 300 with a corresponding coordinate location in the X-direction and the Y-direction. In other words, each element 326 in the surface-rendering, which in this case is the perspective view of the plane 320, represents the data acquired for the location in the plane 300 with the corresponding coordinate location in the X-direction and the Y-direction. Since the elements 326 in the perspective view of the plane 320 correspond to the locations 306 in the plane 300, it should be appreciated that FIG. 3 only includes a subset of the total number of element in the perspective view of the plane 320. According to many embodiments, the perspective view of the plane extends further in both the X-direction 322 and the Y-direction 324.

As discussed previously, generating a surface-rendering may include performing one or more techniques such as shading to calculate a perspective view of the surface defined by the three-dimensional locations of the surface data points. Each element may be assigned a color and/or a grey-scale value. Each element may be represented as one or more pixles in the surface-rendering 320. The surface-rendering process allows a viewer to view the surface at any arbitrary view angle; in other words, a clinician may adjust the view direction from which they view the perspective view of the plane 320. The clinician may, for instance, rotate the surface-rendering about any axis to adjust the view direction.

FIG. 3 also includes a schematic representation of a technique for surface-rendering elements to represent values from multiple different imaging modes in accordance with an embodiment in a display 350. Display 350 allows for the visualization of two different modes of ultrasound imaging data acquired for each location 306 in the plane 300. As in the perspective view of the plane 320, each of the elements in the display 350 corresponds to a location 306 in the plane 300 based on the respective coordinate locations in the plane 300 and the display 350. The display 350 includes an X-direction 352, a Y-direction 354, and a Z-direction 356. Elements in the display 350 correspond to locations 306 in the plane with the same coordinate locations in the X-direction and the Y-direction. For instance, the first location 308 corresponds with a first element 360; the second location 310 corresponds with the second element 362; and the third location 312 corresponds with the third element 364. While only three elements are schematically represented in the display 350, it should be appreciated that a complete display would have a element representing every location in the plane 300. Each element may be represented by a single pixel, or each element may be represented by a plurality of elements according to various embodiments.

As described with respect to the perspective view of the plane 320, each element in the display 350 may be assigned a color or a grey-scale value based on the value of a first parameter acquired during the first ultrasound imaging mode. This may be a grey-scale value acquired during a B-mode according to an embodiment. However, the value of a second parameter acquired during a second ultrasound imaging mode may also be represented in the display 350 based on the height of each element in the Z-direction 356. For instance, the first element 360 is at a height 370 above an X-Y plane 374; the second element is at a height 372 above the X-Y plane 374, and the third element 364 is at a height of zero above the X-Y plane 374. The X-Y plane 374 is perpendicular to the Z-direction 356. As the heights of the elements are relative to the other elements, the exact position of the X-Y plane 374 is not critical, but according to an embodiment, the X-Y plane 374 may be positioned at a height of zero in the Z-direction 356. The height of each of the elements is used to represent/display the value of the second parameters acquired during the second ultrasound imaging mode. The display 350 allows for an intuitive way to display both the first plurality of values acquired during the first ultrasound imaging mode and second plurality of values acquired during the second imaging mode at the same time.

Referring to the method 200, shown in FIG. 2, at step 202, the processor 116 controls the probe 106 to acquire first ultrasound data for a plurality of locations within the plane 300 (shown in FIG. 3) in a first ultrasound imaging mode. According to an exemplary embodiment, the first ultrasound data may comprise B-mode ultrasound data. In B-mode, or brightness mode, intensity (amplitude) data is acquired for each of the plurality of locations 306 within the plane 300. The B-mode data may include, for instance, a first plurality of values acquired at the locations 306. Each of the first plurality of values may represent a first parameter according to an embodiment. Each value may be an intensity value acquired for a specific one of the locations 306. As discussed hereinabove, the processor 116 may steer the focusing of the probe 106 to acquire ultrasound data along a plurality of scan lines. The term “first ultrasound data” may refer the ultrasound data acquired along a plurality of scan lines, the term “first ultrasound data” may also refer to the ultrasound data after it has been scan converted from Polar to Cartesian coordinates so each first ultrasound datum corresponds exactly to one of the plurality of locations 306, or the term “first ultrasound data” may correspond to both the ultrasound data acquired along a plurality of scan lines and the ultrasound data after it has been scan converted from Polar coordinates to Cartesian coordinates. It should be appreciated that the plurality of locations 306 are in accordance with an exemplary embodiment and that other embodiments may have a different density of locations, and/or the locations may be positioned in a different arrangement. For example, some embodiments may perform operations on the data in Polar coordinates, so the plurality of locations may be arranged in a manner that is more convenient for Polar operations, such as along the scan lines.

While the first ultrasound data may be B-mode data according to an embodiment, it should be appreciated that the first ultrasound data may be acquired during a different ultrasound imaging mode according to other embodiments. For example the first ultrasound data may be acquired during any of the following, non-limiting list of ultrasound modes: B-mode, which would result in the acquisition of B-mode (or amplitude) data; strain mode, which would result in the acquisition of strain data; color mode, which would result in the acquisition of color data; flow mode, which would result in the acquisition of flow data. A different parameter may be acquired during each of the ultrasound imaging modes.

At step 204, the processor 116 controls the probe 106 to acquire second ultrasound data for the plurality of locations 306 within the plane 300 (shown in FIG. 3). The second ultrasound data is acquired while in a second ultrasound imaging mode that is different than the first ultrasound imaging mode. So, according to the exemplary embodiment described above, the second ultrasound imaging mode may comprise any ultrasound imaging mode other than B-mode. According to an exemplary embodiment, the second ultrasound imaging mode may be a strain mode and the second ultrasound data may be strain mode data. In strain mode, strain data is acquired for each of the plurality of locations 306 within the plane 300. The strain may be measured in response to stress applied by an operator, such as a manual palpation, or the strain may be measured in response to an anatomically applied stressor, such as pressure exerted by a subject's heart or diaphragm moving. The strain data may include, for instance, a second plurality of values acquired at the plurality of locations 306. The second plurality of values may include a second parameter that is different than the first parameter. Each value may be a strain value acquired from a specific one of the locations 306. As discussed hereinabove, the processor 116 may steer the focusing of the probe 106 to acquire ultrasound data along a plurality of scan lines. The term “second ultrasound data” may refer the ultrasound data acquired along a plurality of scan lines, the term “second ultrasound data” may refer to the ultrasound data after it has been scan converted from Polar to Cartesian coordinates so each first ultrasound datum corresponds exactly to one of the plurality of locations 306, or the term “second ultrasound data” may correspond to both the ultrasound data acquired along a plurality of scan lines and the ultrasound data after it has been scan converted from Polar coordinates to Cartesian coordinates

At step 206, the processor 116 generates an surface-rendering to represent both the first ultrasound data and the second ultrasound data. Step 206 will be described in accordance with multiple exemplary surface-renderings that may be generated in accordance with various embodiments hereinafter. At step 208, the surface-rendering is displayed on the display device 118.

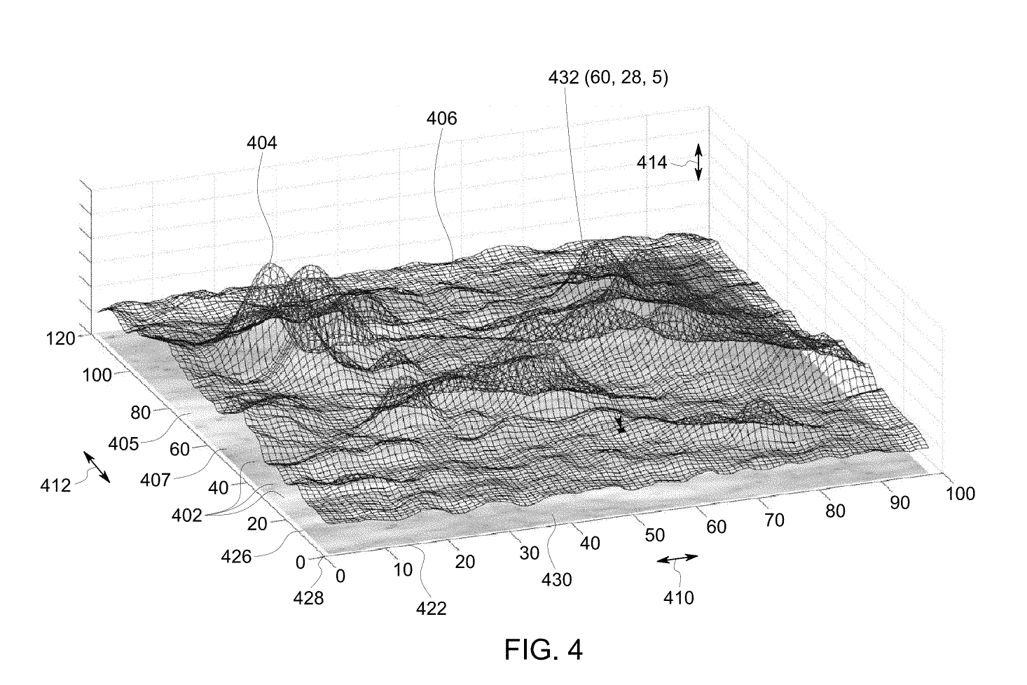

FIG. 4 is a schematic representation of a surface-rendering 400 in accordance with an exemplary embodiment. FIG. 4 includes a plurality of elements 402 and a mesh 404. At step 206, the processor 116 may generate a surface-rendering similar to the surface-rendering 400 shown in FIG. 4. The surface-rendering 400 represents an X-direction 410, a Y-direction 412, and a Z-direction 414. Each of the plurality of elements 402 corresponds to one of the plurality of locations 306 in the plane 300. Each element may be comprised of single pixel or each element may be comprised of a plurality of pixels according to various elements. For example, the each of the plurality of elements 402 may correspond to one of the plurality of locations 306 in the plane 300 in the same manner that the elements 326 in the perspective view of the plane 320 correspond to the plurality of locations 306 in the plane 300. The plurality of elements are arranged to represent a planar surface 405 in the embodiment shown in FIG. 4. The mesh 404 comprises a grid of lines that are evenly spaced in the X-direction 410 and the Y-direction 412. The grid of lines of the mesh 404 are curved, however, to more clearly illustrate height of the surface defined by the mesh 404 in the Z-direction. The mesh 404 is surface-rendered along with the plurality of elements 402 to generate the surface-rendering 400. A coordinate location of a element in the X-direction 410 and the Y-direction 412, which in an exemplary embodiment includes a position in the X-direction 410 and a position in the Y-direction 412 with respect to an origin 428 corresponds to a location in the plane 300 with the same coordinate location in the X-direction 302 and the same position in the Y-direction 304. Using the convention where coordinates of elements/locations are given in (position in X-direction, position in Y-direction), the element 422 may be identified by the coordinate location (14, 0) and the element 426 may be identified by the coordinate location (0, 10). Likewise, the location in the plane corresponding to the first element may be identified by the coordinate location (14, 0) and the location in the plane corresponding to the second element may be identified by the coordinate location (0, 10).

According to the exemplary embodiment discussed above, each element 402 may represent B-mode data, and collectively, the plurality of elements 402 may be displayed as a perspective view (generated through a surface-rendering process) of a B-mode image 430. A B-mode image would conventionally be displayed as a two-dimensional image on a flat display. However, according to the embodiment show in FIG. 4, the perspective view of the B-mode image 430 is generated by changing a view direction with respect to the B-mode image. In a conventional display, a B-mode image would typically be viewed with a 90 degree viewing angle. In other words a viewer would be viewing the plane at a 90 degree angle. However, in the embodiment shown in FIG. 4, the view direction with respect to the X-Y plane 407 is approximately 60 degrees.

FIG. 4 also includes the mesh 404 that defines the non-planar surface 406. The mesh 404 is used to show relief, or elevation, in the Z-direction 414 at specific coordinate locations in the X-Y plane 407. The mesh 404 comprises lines that, at a height of zero in the Z-direction 414, may outline the individual elements 402, the lines of the mesh may outline groups of elements 402 or the lines of the mesh 404 may be evenly spaced in the X-direction 410 and the Y-direction 412. As discussed previously, the lines of the mesh 404 are curved to define the non-planar surface 406 and to show relief, or elevation, in the Z-direction 414. An operator may visually follow the lines of the mesh 406 to understand the various heights of the mesh 406 at particular coordinate locations in the X-direction and the Y-direction.

According to the embodiment shown in FIG. 4, all of the values represented by the height of the mesh 406 in the Z-direction 414 are positive or zero. However, in other embodiments, the non-planar surface 406 defined by the mesh may be used to represent negative values as well, if it is permissible to have negative values for the parameter being measured within a specific ultrasound imaging mode.

For each location in the surface-rendering 400, the grey-scale values of the elements 402 represent the first ultrasound data (intensity values, according to an embodiment where the first ultrasound data is B-mode data). And, the height of the non-planar surface 406 defined by the mesh 404 represents the second ultrasound data, which may be strain data according to an embodiment. The first ultrasound data may include values of a first parameter and the second ultrasound data may include values of a second parameter that is different than the first parameter. A user may use the perspective view of the B-mode image 430 for orientation with respect to anatomical structures within a subject's body, and then the user may determine the value of the second parameter acquired in the second ultrasound imaging mode based on the height of the non-planar surface 406 defined by the mesh 404. The differences in height in the Z-direction 414 provided by the non-planar surface 406 defined by the mesh 404 makes it extremely easy for the user to quickly identify regions with local maximums. Additionally, the non-planar surface 406 defined by the mesh 404 makes it much easier for the user to discern between two areas with relatively similar values. Using the mesh 404 to graphically show the values of the second parameter with respect to a Z-direction should allow for users to assess the information more quickly compared to conventional techniques. Additionally, according to an embodiment, the user may manually adjust a scale in the Z-direction 414 to compress or expand the displayed height of the surface defined by the mesh 404. Therefore, for situations where the values represented by the mesh are all similar, the user may change the scaling in the Z-direction 414 to expand or compress the surface-rendering 400 in the Z-direction 414.

According to an embodiment, the user may position a cursor 432 anywhere on the non-planar surface 406 defined by the mesh 404. The processor 116 may cause the coordinate location and the value of the second ultrasound data for that specific coordinate location to be displayed. For example, the cursor 432 may be represented by a specific color. In response to positioning the cursor 432 on a specific location on the mesh 404, the information (60, 28, 5) is displayed on the display device or, in other embodiments, adjacent to the highlighted portion of the mesh 404. The information (60, 28, 5) represents (position in X-direction, position in Y-direction, and value of second ultrasound datum at the position in the X-direction and the Y-direction).

According to other embodiments, the user may input a command for the processor 116 to locate a maximum value, a minimum value, a local maximum value, or a local minimum value. In response to this, the processor 116 may automatically position the cursor 432 at the respective maximum value, minimum value, local maximum value, or local minimum value and optionally display the information in the form (position in X-direction, position in Y-direction, and value of second ultrasound datum at the coordinate location defined by the position in the X-direction and the position in the Y-direction). According to an embodiment, the cursor 432 may move along the surface defined by the mesh 404 in response to user inputs. According to other embodiments, the cursor may be depicted by highlighting a vertical column from a element or elements 402 in the planar surface 406 to the non-planar surface 406 defined by the mesh 404.

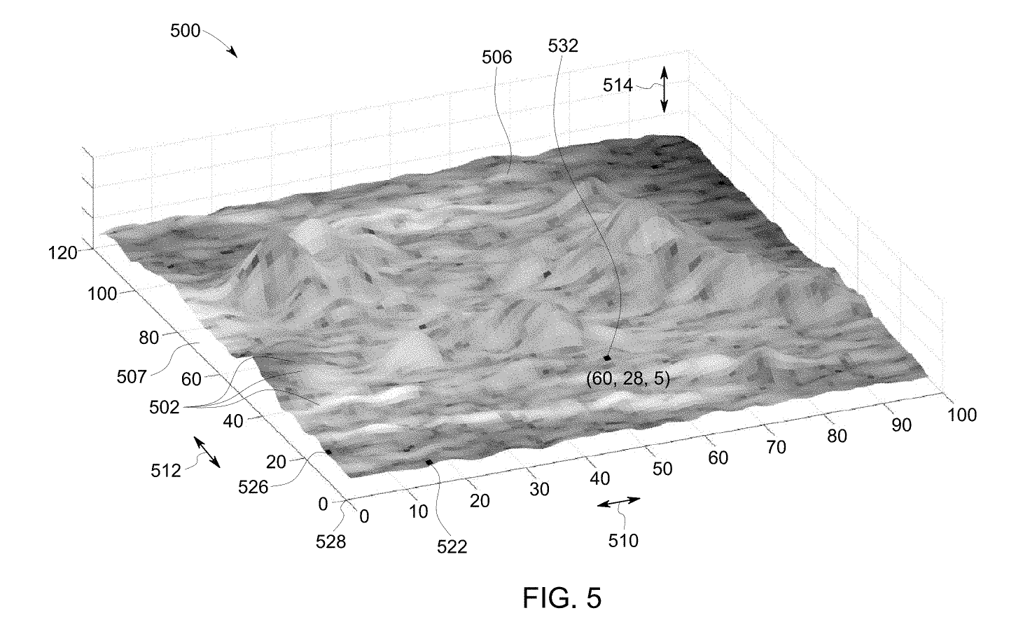

FIG. 5 is an schematic representation of a surface-rendering 500 in accordance with an exemplary embodiment. FIG. 5 includes a plurality of elements 502 that defines a non-planar surface 506. According to an embodiment, at step 206, the processor 116 may generate a surface-rendering similar to the surface-rendering 500 shown in FIG. 5. The surface-rendering 500 represents an X-direction 510, a Y-direction 512, and a Z-direction 514. An X-Y plane 507 is perpendicular to the Z-direction 514 and positioned at a height of zero in the Z-direction 514 Each of the plurality of elements 502 corresponds to one of the plurality of locations 306 in the plane 300. Each element may be comprised of one pixel or a plurality of pixels according to various embodiments. A coordinate location of a element in the X-direction 510 and the Y-direction 512, which in an exemplary embodiment includes a position in the X-direction 510 and a position in the Y-direction 512 with respect to an origin 528 of the surface-rendering 500 corresponds to a location in the plane 300 with the same coordinate location in the X-direction and the Y-direction. Using the convention where coordinate locations of elements/locations in the surface-rendering 500 are given in (position in X-direction, position in Y-direction), the element 522 may be identified by the coordinate location (14, 0) and the element 526 may be identified by the coordinate location (0, 10). Likewise, the location in the plane 300 corresponding to the first element may be identified by the coordinate location (14, 0) and the location in the plane corresponding to the second element may be identified by the coordinate location (0, 10).

According to the exemplary embodiment discussed above, each element 502 may represent B-mode data. In other words, the grey-scale value assigned to each of the elements 502 may be based on B-mode data acquired with the ultrasound imaging system 100. According to other embodiments, each of the elements 502 may be assigned a color based on the first ultrasound data.

In FIG. 5, the elements 502 are positioned at different heights in the Z-direction 514. The height of the non-planar surface 506 (which is defined by the elements 502 according to the embodiment in FIG. 5) in the Z-direction 514 is used to represent the second ultrasound data.

According to the embodiment shown in FIG. 5, all of the values represented by the height of the non-planar surface 506 in the Z-direction 414 are positive or zero. However, in other embodiments, the non-planar surface 506 may be used to represent negative values as well.

For each coordinate location in the surface-rendering 500, the grey-scale values of the elements 502 represent the first ultrasound data (intensity values according to an embodiment where the first ultrasound data is B-mode data). And, the height of the non-planar surface 506 represents the second ultrasound data, which may be strain data according to an embodiment. Those skilled in the art will appreciate that generating the surface-rendering 500 results in a warping of information that is normally displayed as a 2D image. In other words, the grey-scale values (or color values, according to other embodiments) of the elements would conventionally be displayed as pixels in a 2D image. However, the surface-rendering introduces a variable height in the Z-direction 514 to convey second ultrasound data that was acquired in the second ultrasound imaging mode. As such, the surface-rendering 500 represents a novel way to display ultrasound data acquired with two different ultrasound imaging modes at the same time. As noted previously, since the surface-rendering 500 includes a relative offset between the various elements in the Z-direction 514, the resulting surface-rendering 500 appears to be warped compared to a conventional 2D display of the element data (i.e., the first ultrasound data). The user may, however, still use the representation of the first ultrasound data for an understanding of the location with respect to the subject. For example, if the first ultrasound data is B-mode data, then the elements 502 collectively form a warped B-mode image. The user may still use the grey-scale values of the elements 502 to identify anatomical landmarks. The user may then quickly and easily discern the values of the second ultrasound data at each coordinate location in the X-direction 510 and the Y-direction 512 based on the height of the non-planar surface 506 defined by the elements 502. The differences in heights in the Z-direction 514 provided by non-planar surface 506 makes it extremely easy for the user to quickly identify regions/areas with local maximums. Additionally, the non-planar surface 506 defined by the elements 502 also makes it very easy for the user to discern between two areas with relatively similar values. Using the height of the elements in the Z-direction 514 to graphically show the values of the second ultrasound data provides a quick understanding of the data and should allow a clinician to assess the information more quickly when compared to conventional techniques, such as using colors to represent strain values. Additionally, according to an embodiment, the user may manually adjust the scale in the Z-direction 514. Therefore, for situations where the values represented by the height of the non-planar surface 506 are all similar, the user may change the scaling in the Z-direction 514 to expand or compress the surface-rendering 500 in the Z-direction 514.

According to an embodiment, the user may position a cursor anywhere on the non-planar surface 506. The processor 116 may cause the coordinate location and the value of the second ultrasound data for that specific coordinate location to be displayed. For example, a cursor 532 is represented by a specific color. The cursor 532 may be indicated on the surface-rendering 500 with a color or by highlighting the element or elements where the cursor 532 may be currently located on the surface-rendering 500. In response to positioning the cursor 532 on a specific location on the non-planar surface 506, the information (60, 28, 5) is displayed on the surface-rendering adjacent to the highlighted portion of the non-planar surface 506. The information (60, 28, 5) represents (position in X-direction, position in Y-direction, and value of second ultrasound datum at the coordinate location defined by the position in the X-direction and the position in the Y-direction).

According to other embodiments, the user may input a command for the processor 116 to locate a maximum value, a minimum value, a local maximum value, or a local minimum value. In response to this, the processor 116 may automatically position the cursor 532 at the respective maximum value, minimum value, local maximum value, or local minimum value and optionally display the information in the form (position in X-direction, position in Y-direction, and value of second ultrasound datum at the coordinate location defined by the position in the X-direction and the position in the Y-direction). According to an embodiment, the cursor 532 may move along the non-planar surface 506 defined by the elements 502 in response to user inputs.

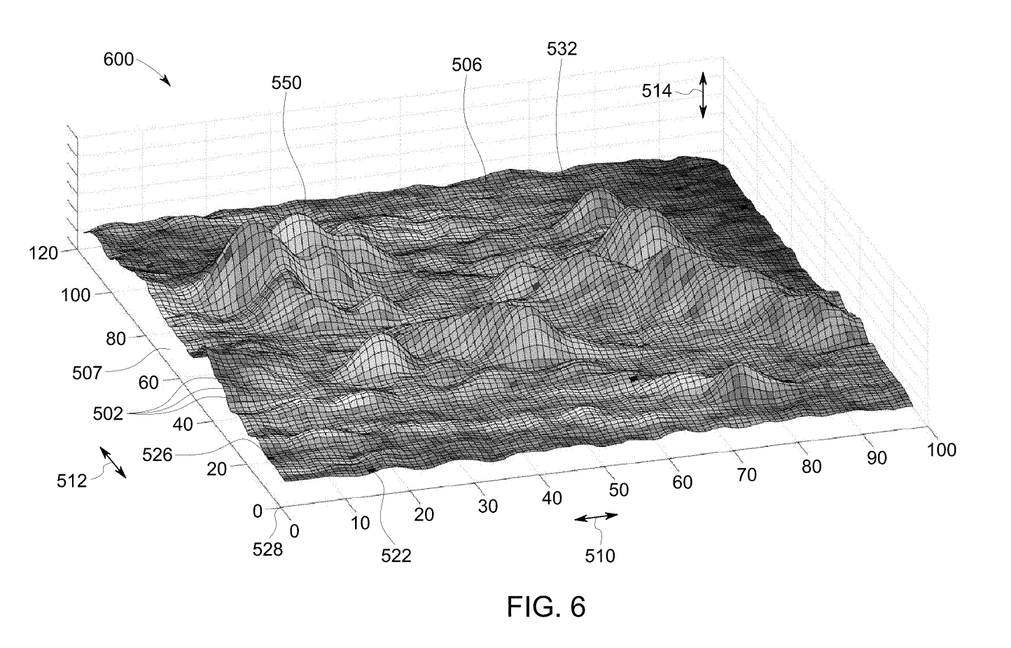

FIG. 6 is an schematic representation of a surface-rendering 600 in accordance with an exemplary embodiment. Many of the elements depicted in FIG. 6 are the same as those that were previously described with respect to FIG. 5. Common reference numbers are used to identify identical elements between FIGS. 5 and 6.

The surface-rendering 600 is a schematic representation of a surface-rendering that may be generated at step 306 according to an embodiment. In addition to the elements that were described with respect to FIG. 5, the surface-rendering 600 also includes a mesh 550. The mesh 550 may outlines each of the elements 502 in the surface-rendering 600 and is used to help more clearly define the non-planar surface 506. According to other embodiments, the spacing of the mesh 550 may be adjusted differently. Instead of outlining each element, the mesh 550 may surround groups of elements of a fixed size/orientation or the mesh may have fixed spacing that is independent of the element size. For example, the mesh 550 may be rendered so that the lines have a fixed spacing in the X-direction 510 and a fixed spacing in the Y-direction 512. The mesh 550 may make it easier for a user to quickly interpret the relative height of each element, or the non-planar surface 506 defined by the elements 502 and the mesh 550.

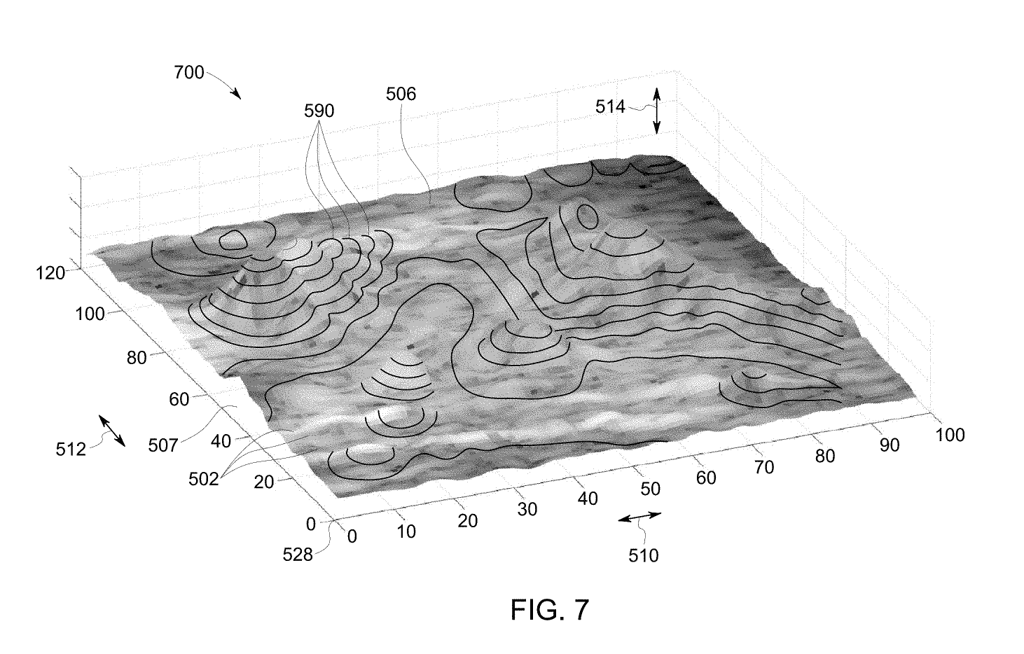

According to other embodiments the surface-rendering may include a plurality of contour lines instead of a mesh to help visually convey height in a Z-direction. FIG. 7 is a schematic representation of a surface-rendering 700 in accordance with an embodiment. Many of the elements represented in FIG. 7 are the same as the elements that were previously described with respect to FIG. 5. Common reference numbers are used to describe identical elements between FIGS. 5 and 7. In addition to the plurality of elements 502, the surface-rendering 700 also includes a plurality of contour lines 590. Each of the contour lines 590 connects points on the non-planar surface 506 that are at the same height in the Z-direction 514. The contour lines 590 are rendered according to a surface-rendering technique, so that the contour lines 590 appear to be spaced at different heights in the Z-direction. The spacing between adjacent contour lines may be fixed at a preset distance, or the spacing between adjacent contour lines may be user adjustable.

According to other embodiments, contour lines may be used to connect locations on the non-planar surface 506 defined by a mesh that are at a same height in a Z-direction. For example, contour lines, such as those depicted in FIG. 7, may be superimposed on top of the surface-rendering 400 shown in FIG. 4 or the surface-rendering 600 shown in FIG. 6. The contour lines may be represented in a different color than the mesh in order to more clearly distinguish between lines of the mesh and the contour lines.

According to another embodiment, a surface-rendering may include contour lines, such as the contour lines 590, superimposed over a perspective view of a plane, such as the perspective view of the plane 405 shown in FIG. 4. The contour lines may still be rendered using a surface-rendering technique to appear as if they are spaced apart in a Z-direction that is perpendicular to the perspective view of the plane. The contour lines would define the surface to illustrate the values of the second ultrasound data.

For any of the embodiments discussed hereinabove, the surface-renderings may be manipulated by the user. For example, the user may adjust the view direction. The user may adjust one or more of a scale in the Z-direction, a scale in the X-direction, and a scale in the Y-direction in order to zoom in or expand on various features. The user may, according to an embodiment, view a cut-plane through the surface-rendering. The cut-plane is a two-dimensional slice that may be positioned at any position and orientation with respect to the surface-rendering. The user may use a cut-plane, for instance, to view a cross-section of the surface-rendering. Displaying both first ultrasound data and second ultrasound data as a surface-rendering provides the user with an easy-to-understand visual representation of the parameter values associate with both ultrasound imaging modes at the same time and provides the user with the flexibility to easily adjust the surface-rendering to emphasize to desired portions of the data.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of displaying data acquired with multiple ultrasound imaging modes, the method comprising:
acquiring first ultrasound data for a plurality of locations within a plane while in a first ultrasound imaging mode, the first ultrasound data comprising a first plurality of values;
acquiring second ultrasound data for the plurality of locations within the plane while in a second ultrasound imaging mode that is different than the first ultrasound imaging mode, the second ultrasound data comprising a second plurality of values;
generating a surface-rendering based on both the first ultrasound data and the second ultrasound data, the surface-rendering comprising a non-planar surface and representing an X-direction, a Y-direction, and a Z-direction, where the first ultrasound data is represented by one of a plurality of colors and a plurality of grey-scale values in the surface-rendering, and where the second ultrasound data is represented by a plurality of heights of the non-planar surface in the Z-direction of the surface-rendering; and
displaying the surface-rendering on a display device.

2. The method of claim 1, where the surface-rendering further comprises a planar surface.

3. The method of claim 2, where the non-planar surface is defined by a mesh.

4. The method of claim 1, where planar surface is perpendicular to the Z-direction.

5. The method of claim 4, where the non-planar surface is defined by a mesh.

6. The method of claim 5, where the surface-rendering comprises the mesh superimposed over the planar surface.

7. The method of claim 2, where the surface-rendering further comprises a plurality of contour lines, where each of the contour lines connects a plurality of locations on the non-planar surface with a same height in the Z-direction.

8. The method of claim 5, where the surface-rendering further comprises a plurality of contour lines displayed on the mesh, where each of the contour lines connects a plurality of locations on the mesh with a same height in the Z-direction.

9. The method of claim 2, where the first ultrasound imaging mode comprises a B-mode and where the planar surface comprises a perspective view of a B-mode image.

10. The method of claim 9, where the second ultrasound imaging mode comprises an ultrasound mode selected from the list consisting of: a strain mode, a flow mode, and a color mode.

11. The method of claim 1, further comprising adjusting a view direction of the surface-rendering in response to a user input.

12. The method of claim 11, where generating the surface-rendering occurs in real-time as the first ultrasound data and the second ultrasound data are acquired.

13. An ultrasound imaging system comprising:
a probe;
a display device; and
a processor in electronic communication with the probe and the display device, wherein the processor is configured to:
control the probe to acquire first ultrasound data for a plurality of locations within a plane while in a first ultrasound imaging mode, the first ultrasound data comprising a first plurality of values;
control the probe to acquire second ultrasound data for the plurality of locations within the plane while in a second ultrasound imaging mode that is different than the first ultrasound imaging mode, the second ultrasound data comprising a second plurality of values;
generate a surface-rendering to represent both the first ultrasound data and the second ultrasound data, the surface-rendering comprising a non-planar surface and representing an X-direction, a Y-direction and a Z-direction, where each of the plurality of locations is represented by a coordinate location in the surface-rendering in the X-direction and the Y-direction, where the first ultrasound data is represented by one of a plurality of color values and a plurality of grey-scale values in the surface-rendering, and the second ultrasound data is represented by a plurality of heights of the non-planar surface in the Z-direction of the surface-rendering; and
display the surface-rendering on the display device.

14. The ultrasound imaging system of claim 13, wherein the processor is further configured to generate the surface-rendering in real-time as the first ultrasound data and the second ultrasound data are being acquired.

15. The ultrasound imaging system of claim 13, where the non-planar surface represents both the first ultrasound data and the second ultrasound data, where the non-planar surface comprises a plurality of elements, each element positioned at a unique coordinate location in the X-direction and the Y-direction, where each of the plurality of elements is colorized with one of the plurality of colors to represent one of the plurality of first values acquired for a corresponding coordinate location in the X-direction and the Y-direction within the plane, and each of the plurality of elements is positioned at a height in the Z-direction to represented one of the plurality of second values acquired for a corresponding coordinate location in the X-direction and the Y-direction within the plane.

16. The ultrasound imaging system of claim 13, where the non-planar surface represents both the first ultrasound data and the second ultrasound data, where the non-planar surface comprises a plurality of elements, each element positioned at a unique coordinate location in the X-direction and the Y-direction, where each of the plurality of elements is assigned one of the plurality of grey-scale values to represent one of the plurality of first values acquired for a corresponding coordinate location in the X-direction and the Y-direction within the plane, and each of the plurality of elements is positioned at a height in the Z-direction to represented one of the plurality of second values acquired for a corresponding coordinate location in the X-direction and the Y-direction within the plane.

17. The ultrasound imaging system of claim 13, wherein the surface-rendering comprises a mesh defining the non-planar surface.

18. The ultrasound imaging system of claim 13, where the surface-rendering comprises a planar surface that is perpendicular to the Z-direction, where the planar surface represents the first ultrasound data.

19. The ultrasound imaging system of claim 18, where the planar surface is a perspective view of a B-mode image.

20. The ultrasound imaging system of claim 13, wherein the surface-rendering further comprises a plurality of contour lines, where each of the plurality of contour lines connects a plurality of locations on the non-planar surface with a same height in the Z-direction.

21. The ultrasound imaging system of claim 17, wherein the surface-rendering further comprises a plurality of contour lines, where each of the plurality of contour lines connects a plurality of locations on the mesh with a same height in the Z-direction.
